(19) United States
(12) Patent Application Publication
(10) Pub. No.: US 2007/0167528 A1
(43) Pub. Date: Jul. 19, 2007

(54) Methods of treating hepatocellular carcinoma with denspm

(75) Inventors: David Davidson, Lincoln, MA (US); Raymond Bergeron, Gainesville, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: Genzyme Corporation, Cambridge, MA (US); University of Florida Research Foundation, Gainesville, FL (US)

(21) Appl. No.: 11/548,000

(22) PCT Filed: Apr. 12, 2005
(86) PCT No.: PCT/US05/12039

§ 371 Date: Oct. 10, 2006

Related U.S. Application Data
(60) Provisional application No. 60/561,438, filed on Apr. 12, 2004.

(51) Int. Cl. A61K 31/13

(52) U.S. Cl. 514/674

(57) ABSTRACT

The disclosure provides methods for treating hepatocellular carcinoma. The methods include administering to a subject an effective amount of DENSPM to treat the hepatocellular carcinoma.

This application claims priority to U.S. Patent Application No. 60/561,438, filed Apr. 12, 2004, which is incorporated herein by reference in its entirety.

### DESCRIPTION OF THE INVENTION

1. Field of the Invention

The invention relates to the use of DENSPM in methods of treating hepatocellular carcinoma.

2. Background of the Invention

Hepatocellular carcinoma (HCC) is relatively uncommon in developed areas of the world. However, it affects approximately one million people each year and represents the fifth most common neoplasm worldwide (Alsowmely, A. M., et al. (2002) Aliment Pharmacol. Ther. 16(1): 1-15). The American Cancer Society estimates that 17,300 new cases of liver cancer were diagnosed in the United States in 2003, of which approximately two-thirds are HCC. In this same year, 14,400 deaths from liver cancer are expected, reflecting the absence of successful therapies for these tumors.

To date, other than curative resection, treatments for HCC have had minimal impact on survival. Unfortunately, approximately 90% of HCC patients have unresectable HCC. Moreover, even after potentially curative hepatectomy in patients with resectable HCC, new HCC arises in the cirrhotic remnants in 70% of these patients, and frequently arises in the grafted liver following orthotropic liver transplantation. Other approaches to treating HCC, such as intralesional ethanol injection, chemoembolization, radiofrequency ablation, cryosurgery and radiation therapy have demonstrated success in selected patient populations. However, the efficacies of these approaches have not been definitively established. Both percutaneous intralesional ethanol injection and transarterial chemoembolization have shown limited success, but not without risks of serious side effects. Radiotherapy is not usually an option because the liver is very radiosensitive. Systemic chemotherapy for HCC has had uniformly poor response rates, and therefore it is generally reserved for patients ineligible for curative resection.

All systemic therapies for HCC to date are associated with uniformly poor outcomes, and no chemotherapeutic agent, alone or in combination with other treatments, has been associated with any improvement in survival rates (Fuchs, C. S., et al. (2002) Cancer 94(12): 3186-91). In addition, most patients with HCC have underlying liver disease so their ability to tolerate chemotherapy regimens or undergo surgical therapy is often compromised.

In view of the poor treatment choices available, there is a clear need for new regimens to treat hepatocellular carcinoma.

### SUMMARY OF THE INVENTION

The invention provides methods for treating hepatocellular carcinoma. Additional aspects of the invention will be set forth in part in the following description, in part will be understood by the description, and in part may be learned by practice of the invention.

In one embodiment, the invention provides methods for treating hepatocellular carcinoma comprising administering a therapeutically effective amount of DENSPM or a pharmaceutically-acceptable salt thereof to a patient in need of such treatment. DENSPM or a pharmaceutically-acceptable salt thereof may be administered intravenously, intralesionally or intrahepatically.

In some embodiments of the invention, patients with unresectable hepatocellular carcinoma are treated. In other embodiments, patients having hepatocellular carcinoma that is refractory to other methods of treatment are treated.

In some embodiments of the invention, DENSPM is administered once daily. In other embodiments, DENSPM is administered two, three, four, or more times a day, or is administered continuously as an infusion.

In some embodiments of the invention, DENSPM is administered at a dose ranging from about 30 to about 300 mg/m², inclusive. In some of these embodiments, DENSPM is administered in doses of 30, 60, 90, 120, 150, 180, 210, 240, 270, 300 mg/m², or in doses of any increment in between. For example, DENSPM may be administered at a dose varying from 30 to 60 mg/m², 90 to 120 mg/m², 120 to 150 mg/m², 150 to 180 mg/m², 180 to 210 mg/m², 210 to 240 mg/m², 240 to 270 mg/m², and 270 to 300 mg/m².

In some embodiments of the invention described above, DENSPM itself is administered. In other embodiments described above, pharmaceutically acceptable salts of DENSPM are administered. Preferred pharmaceutically acceptable salts of DENSPM include DENSPM hydrochloride.

In some embodiments of the invention, DENSPM is administered to patients who are not receiving any other therapy for HCC. In some of these embodiments, the patients are receiving other therapy for HCC but are not receiving any other anticancer agents to treat HCC.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention as claimed.

### DESCRIPTION OF THE EMBODIMENTS

In order that the present invention may be more readily understood, certain terms are defined herein. Additional definitions are set forth throughout the detailed description.

The terms “hepatocellular carcinoma” and “HCC” are used interchangeably herein to refer to cancer that arises from hepatocytes, the major cell type of the liver.

The term “DENSPM,” as used herein, refers to N¹, N¹¹-diethylnorspermine, which is N,N′-Bis[3-(ethylamino)propyl],1,3-propanediamine. Unless explicitly stated otherwise, the term DENSPM includes DENSPM and any pharmaceutically acceptable salts thereof. A preferred salt of DENSPM is N,N′-Bis[3-(ethylamino)propyl],1,3-propanediamine tetrahydrochloride. DENSPM is a known dysfunctional polyamine analogue and may be prepared using art-known methods. For example, Bergeron describes the synthesis of polyamines such as DENSPM, in U.S. Pat. Nos. 5,091,596; 5,342,945; and 5,866,613, as well as J. Med. Chem. 1988, 31(6):1183-90, each hereby incorporated by reference.

The term “pharmaceutically-acceptable salt,” as used herein, includes salts that may be safely administered to a patient. Preferred pharmaceutically-acceptable salts of DENSPM include, but are not limited to, those prepared from the following acids: hydrochloric, hydrobromic, sulfuric, nitric, phosphoric, malaic, acidic, salicylic, p-toluene sulfonic, tartaric, citric, methane dysphonic, formic, malonic, succinic, naphthlane-two-sulfonic and benzene sulfonic. Pharmaceutically-acceptable salts can also be prepared from metals such as alkaline metal or alkaline earth metals, such as, sodium, potassium, and calcium, magnesium, and zinc. Pharmaceutically acceptable salts also include but are not limited to salts formed with acids such as sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, 2-butyne-1,4-dioate, 3-hexyne-2,5-dioate, benzoate, chlorobenzoate, hydroxybenzoate, methoxybenzoate, phthalate, xylenesulfonate, phenylacetate, phenylpropionate, phenylbutyrate, citrate, lactate, hippurate, B-hydroxybutyrate, glycollate, maleate, tartrate, methanesulfonate, propanesulfonate, naphthalene-1-sulfonate, naphthalene-2-sulfonate, and mandelate.

The terms “treatment,” “therapeutic method,” and their cognates refer to treatment of existing conditions as well as prophylactic or preventative measures. Those in need of treatment may include individuals already having HCC, as well as those who are at risk for or who may ultimately acquire HCC.

The terms “effective dose,” “amount effective,” “effective amount,” and their cognates, as used herein, refer to the amount of DENSPM that provides a benefit in the treatment and/or management of HCC. Effective amounts can be determined by methods well-known in the art or, e.g., as described in the Examples. In one embodiment of the invention, an effective amount for treating hepatocellular carcinoma is that amount necessary to inhibit the growth of or diminish the size of an HCC tumor in a patient. When administered to a patient, effective amounts depend on the severity of the HCC; individual patient parameters, such as physical condition, size, and weight; concurrent treatment, if any; frequency of treatment; and the mode of administration. Concomitant therapy, if any, may also affect the effective amount. It is generally preferred to administer a dose ranging from the therapeutic level up to the maximum tolerated dose.

The “maximum tolerated dose,” or “MTD,” as used herein, refers to the largest dose of DENSPM that an adult patient can be administered with safety to treat HCC. The maximum tolerated dose may be determined by those skilled in the art with no more than routine experimentation.

The term “therapeutic level,” as used herein, means the minimum concentration of DENSPM that is therapeutically effective in a particular patient. Of course, one of skill in the art will recognize that the therapeutic level and MTD may vary depending on the individual being treated and the severity of the condition. For example, the age, body weight, and medical history of the individual patient may affect the therapeutic efficacy of the therapy. A competent physician can consider these factors and adjust the dosing regimen without undue experimentation to ensure the dose is achieving the desired therapeutic outcome. It is also noted that the clinician and/or treating physician will know how and when to interrupt, adjust, and/or terminate therapy in conjunction with individual patient response.

The term “administer” and its cognates, as used herein, refers to the act of giving DENSPM to a patient. A variety of administration routes are available. A particular administration route will depend on the severity of the HCC, as well as other factors, such as, the convenience to the physician and/or patient. The methods of this invention, generally, may be practiced using any mode of administration that is medically acceptable, meaning any mode that produces effective levels of DENSPM without causing clinically unacceptable adverse effects. Administration modes includes oral, rectal, sublingual, topical, buccal, nasal, rectal, vaginal, transdermal, and parenteral routes of administration. The term “parenteral,” as used herein, encompasses subcutaneous, intravenous, bolus injection, intraperitoneal, intramuscular, and infusion routes of administration. Intravenous routes of administration are preferred.

The terms “subject” and “patient” are used interchangeably herein to mean humans, non-human primates, dogs, cats, horses, sheep, goats, cows, rabbits, pigs and rodents. Human subjects are preferred.

As used herein, the term “unresectable hepatocellular carcinoma,” means HCC that cannot be cured using standard surgical techniques, for example, by partial hepatectomy or by orthotropic liver transplantation.

As used herein, the term “refractory” means a disorder, e.g., HCC, that is resistant to treatment. Patients with HCC that is refractory to other methods of treatment include patients that do not respond positively to standard approaches to treating HCC, such as intralesional ethanol injection, chemoembolization, radiofrequency ablation, prior surgery, radiation therapy, or chemotherapy.

The term “pharmaceutically acceptable carrier,” as used herein, means one or more compatible solid or liquid fillers, diluants or encapsulating substances which are suitable for administration to a patient. The term “carrier” denotes an organic or inorganic ingredient, natural or synthetic, with which the active ingredient DENSPM is combined to facilitate the application.

The terms “pharmaceutical composition” and “pharmaceutical preparation” are used interchangeably herein to mean DENSPM in association with a pharmaceutically-acceptable carrier. Excipients and/or adjuvants may optionally be added to the pharmaceutical preparations. DENSPM preparations may be conveniently provided in unit dosage form and may be prepared by methods well-known in the art of pharmacy. Formulations suitable for intravenous administration are preferred. Due to its relatively high solubility, DENSPM as its hydrochloride salt may be dissolved in sterile water for injection (WFI). Such a solution may be filled into individual dose vials and lyophilized for future reconstitution and administration.

Examples of suitable aqueous and nonaqueous excipients include water, ethanol, polyols (such as glycerol, propylene glycol, polyethylene glycol, and the like), oils, injectable organic esters, and mixtures thereof. Proper fluidity can be maintained, for example, by the use of surfactants.

The compositions may also contain adjuvants such as preservatives, wetting agents, emulsifying agents, and dispersing agents. Prevention of the action of microorganisms may be achieved by the inclusion of various antibacterial and/or antifungal agents, for example, paraben, chlorobutanol, phenol sorbic acid, and the like. It also may be desirable to include isotonic agents, such as sugars, sodium chloride, and the like in the compositions.

For parenteral administration, such as intravenous administration, pharmaceutical compositions comprising DENSPM may be formulated as isotonic suspensions, solutions, or emulsions, in oily or aqueous vehicles, and may contain formulatory agents such as suspending, stabilizing, and/or dispersing agents. Alternatively, the compositions may be provided in dry form such as a powder, crystalline, or freeze-dried solid, for reconstitution with a liquid such as a sterile pyrogen-free water or isotonic saline before use. They may be provided for use in sterile ampoules or vials.

Anti-cancer therapeutics are often given in combination with other types of chemotherapy, including other chemotherapeutic agents. In a preferred embodiment, DENSPM is administered to patients with HCC who are not receiving any other anti-cancer agents. These patients may optionally receive other anti-cancer regiments, such as radiation therapy.

The Examples illustrate an embodiment of the invention wherein DENSPM is administered to patients with HCC, as well as how therapeutic doses and the maximum tolerated dose are determined.

Other embodiments of the invention will be apparent to those skilled in the art from consideration of the specification and practice of the invention disclosed herein. It is intended that the specification and examples be considered as exemplary, only, with a true scope and spirit of the invention being indicated by the following claims.

### EXAMPLES

### Example 1

### Treatment Regimen

Day 1 is defined as the first day of treatment. The following baseline assessments typically are performed on Day 1, prior to the first dose of DENSPM: physical exam, vital signs, laboratory tests (e.g., hematology, coagulation, chemistry, and urinalysis), performance status (Karnofsky), adverse event monitoring, and concomitant medication monitoring.

A treatment cycle is 28 days long. During Cycle 1, patients receive DENSPM thrice-weekly as a single 15-minute IV infusion on Mon-Wed-Fri or Tues-Thurs-Sat during the first 12 days of the cycle (e.g., Days 1, 3, 5, 8, 10, and 12). Following Day 12 treatment, patients have 16 days without DENSPM treatment prior to initiating the next treatment cycle.

DENSPM treatments are administered on an outpatient basis during Weeks 1 and 2 of each treatment cycle, on either a Mon-Wed-Fri or Tues-Thurs-Sat schedule. The appropriate dose is withdrawn and further diluted in 50 mL of normal saline and infused intravenously over 15 minutes into a peripheral vein once daily. Patients remain in the clinic for observation for 90 minutes following the administration of DENSPM. Vital signs are taken prior to discharge. Heights and ideal weights are used to calculate surface area. For males, ideal weight is defined as 50 kg+2.3 kg * (each inch of height greater than 60 inches). For females, ideal weight is defined as 45.5 kg+2.3 kg * (each inch of height greater than 60 inches). For individuals having a height of 60 inches or less, ideal weight is defined as 50 kg (males) or 45.5 kg (females).

If the treatment schedule is interrupted, missed doses are made up as long as the lapse in treatment does not exceed five days, in order to achieve a full 6 dose treatment cycle. The start of the next treatment cycle is typically scheduled no sooner than 21 days from the last dose, starting on a Monday or Tuesday. The administration of subsequent cycles of therapy is delayed if a patient fails to fully meet the conditions of recovery prior to initiating a subsequent treatment cycle, in which case the patient is re-evaluated after 7 days. Initiation of subsequent cycles of therapy may be delayed for a maximum of 14 days to allow for full recovery from toxicity. Any patient not meeting the recovery criteria and not able to start treatment within 14 days of the scheduled treatment date may be discontinued from the therapy.

Typically, during the cycle, renal and liver function tests are performed, hematology is assessed, adverse effects are monitored, and concomitant medications are monitored. Renal and liver function tests may be performed post-treatment during the first 12 days of the cycle on Wed-Fri-Mon-Wed-Fri for patients receiving treatment on a Mon-Wed-Fri schedule or Thurs-Sat-Tues-Thurs-Sat for patients receiving treatment on a Tues-Thurs-Sat schedule (e.g., Days 3, 5, 8, 10, 12) and once during the third week of the cycle (e.g., Days 15-19). Additional tests may be performed to follow-up on any abnormal results. Hematology may be assessed on Friday/Saturday (Day 10) during Week 2 and once during the third week of the cycle (e.g., Days 15-19). Additional tests may be performed to follow-up on any abnormal results. Stool guiac tests may be performed if clinically significant reductions are observed for hemoglobin and hematocrit. Adverse events and concomitant medications are typically monitored throughout the 28-day cycle. Since DENSPM is known to transiently and reversibly elevate serum creatinine levels, caution is used when combining other nephrotoxic agents with DENSPM.

All treatment-related toxicities, with the exception of creatinine, typically recover to≦Grade 1 or to baseline for the patient prior to initiating the even-numbered Cycles 2, 4, and 6. Treatment is usually held for a maximum of 14 days for patients experiencing an incidence of serum creatinine greater than or equal to 1.5×ULN (upper limit of normal) until creatinine recovers. For these patients, additional doses are typically dropped by 25% and no dose re-escalations are allowed, as described below.

The following safety assessments are typically performed to assess recovery prior to dosing (or, with the exception of vital signs, within 7 days prior to the first treatment day of the cycle): physical exam, vital signs, and laboratory tests. Patients eligible for treatment based on the recovery criteria described above may receive a subsequent cycle of treatment. Patients may receive 1, 2, 3, 4, 5, 6, 7, 8, 9, 10 or more cycles of treatment.

### Example 2

### DENSPM Formulation

DENSPM may be supplied as a lyophilized powder, for example packaged in 10 mL clear glass vials containing 215 mg. Such a lyophilized powder may be prepared by dissolving DENSPM hydrochloride in sterile WFI to provide a 5% solution. Such solution may be pH adjusted to a pH of preferably between about 5.8 and 6.2, most preferably 6.0, with 2N NaOH. 4.3 ml of this solution is sterile filled into the glass vials thru Millipak (R) 0.45 and 0.22 micron filters and then lyophilized. DENSPM in this form is physically and chemically stable when stored at room temperature. DENSPM is reconstituted with 4.3 mL of water for injection, USP. The resulting solution contains 50 mg/mL of DENSPM and is chemically and physically stable for up to approximately 96 hours at room temperature. When further diluted in 50 mL normal saline, the resulting admixture is chemically and physically stable for up to 24 hours when stored at room temperature. After reconstitution, the drug is normally used within 8 hours because it contains no preservatives.

What is claimed is:

1. A method of treating hepatocellular carcinoma comprising administering to a subject in need thereof an amount of DENSPM or a pharmaceutically-acceptable salt thereof effective to treat the hepatocellular carcinoma.

2. The method of claim 1, wherein the hepatocellular carcinoma is unresectable.

3. The method of claim 1, wherein the hepatocellular carcinoma is refractory to other methods of treatment.

4. The method of claim 1, wherein the DENSPM is administered once daily.

5. The method of claim 1, wherein the DENSPM is administered intravenously.

6. The method of claim 5, wherein the DENSPM is administered at a dose ranging from about 30 to about 300 mg/m².

7. The method of claim 6, wherein the DENSPM is administered at a dose ranging from about 30 to about 60 mg/m².

8. The method of claim 6, wherein the DENSPM is administered at a dose ranging from about 60 to about 90 mg/m².

9. The method of claim 6, wherein the DENSPM is administered at a dose ranging from about 90 to 120 mg/m².

10. The method of claim 6, wherein the DENSPM is administered at a dose ranging from about 120 to 150 mg/m².

11. The method of claim 6, wherein the DENSPM is administered at a dose ranging from about 150 to 180 mg/m².

12. The method of claim 6, wherein the DENSPM is administered at a dose ranging from about 180 to 210 mg/m².

13. The method of claim 6, wherein the DENSPM is administered at a dose ranging from about 210 to 240 mg/m².

14. The method of claim 6, wherein the DENSPM is administered at a dose ranging from about 240 to 270 mg/m².

15. The method of claim 6, wherein the DENSPM is administered at a dose ranging from about 270 to 300 mg/m².

16. The method of claim 1, wherein DENSPM hydrochloride is administered to the patient.

17. The method of claim 16, wherein the DENSPM hydrochloride is administered to the patient intravenously.

18. The method of claim 17, wherein the DENSPM hydrochloride is administered at a dose ranging from about 30 to about 300 mg/m².

19. The method of claim 18, wherein the DENSPM hydrochloride is administered at a dose ranging from about 30 to about 60 mg/m².

20. The method of claim 18, wherein the DENSPM hydrochloride is administered at a dose ranging from about 60 to about 90 mg/m².

21. The method of claim 18, wherein the DENSPM hydrochloride is administered at a dose ranging from 90 to 120 mg/m².

22. The method of claim 18, wherein the DENSPM hydrochloride is administered at a dose ranging from 120 to 150 mg/m².

23. The method of claim 18, wherein the DENSPM hydrochloride is administered at a dose ranging from 150 to 180 mg/m².

24. The method of claim 18, wherein the DENSPM hydrochloride is administered at a dose ranging from 180 to 210 mg/m².

25. The method of claim 18, wherein the DENSPM hydrochloride is administered at a dose ranging from 210 to 240 mg/m².

26. The method of claim 18, wherein the DENSPM hydrochloride is administered at a dose ranging from 240 to 270 mg/m².

27. The method of claim 18, wherein the DENSPM hydrochloride is administered at a dose ranging from 270 to 300 mg/m².

28. A method of treating hepatocellular carcinoma comprising administering to a subject in need thereof a 28-day therapeutic regimen, said regimen comprising administration of DENSPM or a pharmaceutically-acceptable salt thereof on days 1, 3, 5, 8, 10 and 12, followed by 16 days without DENSPM administration to said subject.

29. The method of claim 28, wherein DENSPM is administered as DENSPM hydrochloride.

30. The method of claim 29, wherein the regimen is repeated 1, 2, 3, 4 or 5 times.
